(19) United States
(12) Patent Application Publication
(10) Pub. No.: US 2024/0410000 A1
(43) Pub. Date: Dec. 12, 2024

(54) HLA GENE AMPLIFICATION PRIMER, KIT, SEQUENCING LIBRARY ESTABLISHMENT METHOD, AND SEQUENCING METHOD

(71) Applicant: XI'AN HAORUI GENE TECHNOLOGIES LTD, Xi'an (CN)

(72) Inventors: Bo WANG, Xi'an (CN); Runjun HE, Xi'an (CN); Chaoqiong CHEN, Xi'an (CN); Long YANG, Xi'an (CN); Yue ZHANG, Xi'an (CN)

(21) Appl. No.: 18/701,537

(22) PCT Filed: Apr. 25, 2022
(86) PCT No.: PCT/CN2022/089017

§ 371 Date: Apr. 15, 2024

(30) Foreign Application Priority Data
Oct. 15, 2021 (CN) 202111204073.9

(51) Int. Cl. C12Q 1/6881; C12N 15/10

(52) U.S. Cl. CPC C12Q 1/6881; C12N 15/1093

(57) ABSTRACT

Provided is an HLA gene amplification primer, comprising primers of any one or more groups of the following nine primer groups: a first primer group: SEQ ID NO: 1 and SEQ ID NO: 2; a second primer group: SEQ ID NO: 3 and SEQ ID NO: 4; a third primer group: SEQ ID NO: 5 and SEQ ID NO: 6; a fourth primer group: SEQ ID NO: 7 and SEQ ID NO: 8; a fifth primer group: SEQ ID NO: 9 and SEQ ID NO: 10; a sixth primer group: SEQ ID NO: 11 and SEQ ID NO: 12; a seventh primer group: SEQ ID NO: 12 and SEQ ID NO: 13; an eighth primer group: SEQ ID NO: 14 and SEQ ID NO: 15; and a ninth primer group: SEQ ID NO: 16, SEQ ID NO: 17, and SEQ ID NO: 18. Also provided is a kit for HLA gene sequencing, which comprises the described HLA gene amplification primer; provided is a gene sequencing library construction method based on the described HLA gene amplification primer and an HLA gene sequencing method based on the HLA gene sequencing library construction method.

### CROSS-REFERENCE TO RELATED APPLICATIONS

The present disclosure claims priority to Chinese Patent Application No. 202111204073.9, entitled “HILA gene amplifying primer, kit, sequencing library construction method, and sequencing method”, filed on Oct. 15, 2021, the content of which is incorporated herein by reference in its entirety.

### TECHNICAL FIELD

The present disclosure relates to the field of nucleic acid sequencing technology, specifically to HLA gene amplifying primers, a kit, a sequencing library construction method, and a sequencing method.

### BACKGROUND

Human leukocyte antigen (HLA) molecules present antigens to T lymphocytes for recognition, playing an extremely important role in the adaptive immune response. HLA gene complex, which is a gene cluster encoding human major histocompatibility complex (MHC) molecules, is a highly polymorphic complex composed of a series of closely linked loci and located on the short arm of chromosome 6. The HLA gene complex is the most complex polymorphic system currently known in the human body. HLA is a highly polymorphic allogeneic antigen which, in chemical aspects, is a class of glycoproteins composed of an α-heavy chain (glycosylated) non-covalently combined to a β-light chain. Its peptide chain has an amino-terminal portion extending outward (accounting for about ¾ of the entire molecule), a carboxyl-terminal portion penetrating into the cytoplasm, and a middle hydrophobic portion located within the cell membrane. HLA is divided into class I molecules and class II molecules according to their distribution and function. HLA-class I molecules are presenting molecules for endogenous antigens; HLA-class II molecules are presenting molecules for exogenous antigens. HLA-class I molecules are encoded by HLA-A, B, and C genes, whose specificity depends on the α-heavy chain; their β-light chain is a β2-microglobulin whose encoding gene is located on chromosome 15. HLA-class II molecules are controlled by the HLA-D region (including 5 subregions), the A gene and B gene of each subregion encode the α-heavy chain and β-light chain, respectively, and the antigen polymorphism depends on the β-light chain. All the above genes are polymorphic sites (multiple alleles) and are co-dominant. If MHC is considered as a whole, its polymorphism is even more prominent. It is conservatively estimated that there are at least 1,300 different haplotypes, corresponding to approximately 17×10⁷ genotypes. HLA genes are also an important genetic structure in humans and are widely used in the fields of forensic identification, organ transplantation and match, and so on. It is well known that high polymorphism is a very important feature of HLA genes. Previous studies on polymorphisms of human leukocyte antigen gene mainly focused on exons encoding antigen-binding peptides (exons 2-4 of HLA-A, HLA-B genes, exons 2 of HLA-DQB1 and HLA-DRB1 genes). Many researchers have done a lot of work on typing technology, the detection of polymorphic sites, disease correlation, and evolutionary analysis of these exons. However, more and more studies have shown that polymorphic sites in the non-coding regions of HLA genes and functions thereof also cannot be ignored.

In the early years, human leukocyte antigen (HLA) genotyping was carried out by serology, with disadvantages including possible occurrence of false negative or false positive results, which reduces the accuracy; limited source of highly specific HLA antisera, which makes it difficult to overcome the cross-reaction of monoclonal antiserum; requirement of 7-10 ml of whole blood to prepare viable lymphocytes and even viable B cells for serological typing; difficult collection and preservation of cells. With the progress of technology, DNA typing methods of HLA genes came out. Several common HLA genotyping technologies currently on the market are as follows: Sequence-Specific Oligonucleotides Probes (SSO): Sequence-Specific Primers (SSP); Sequencing-Based Typing (SBT); and Next Generation Sequencing (NGS). SSO has the advantages of simple experiments and short experimental cycles, however, this technology cannot detect new alleles and can only distinguish between known typing sequences, and the requirement of a large number of probes to achieve effective typing is also one of its disadvantages. SSP also has the advantages of simple experiments and short experimental cycles, but it also has the problem of the inability to detect new alleles and distinguish pseudogenes. SBT has the advantage of detecting new alleles, but it cannot perform phasing on the sample DNA and may produce ambiguous results for heterozygote samples, making accurate typing impossible. NOS technology also has the advantage of detecting new alleles, however. it also leads to introducing ambiguous results because the assembly of the original sequence obtained by sequencing is very dependent on data calculation due to the short read length of sequencing, although the typing results can reach a 2-field resolution.

### SUMMARY

Based on this, it is necessary to provide HLA gene amplifying primers, a kit, a sequencing library construction method. and a sequencing method that can improve the accuracy of the results and simplify the process.

According to a first aspect, the present disclosure provides HLA gene amplifying primers, including primers in any one or more of the following nine primer sets:
a first primer set: SEQ ID NO: 1 and SEQ ID NO: 2;
a second primer set: SEQ ID NO: 3 and SEQ ID NO: 4;
a third primer set: SEQ ID NO: 5 and SEQ ID NO: 6;
a fourth primer set: SEQ ID NO: 7 and SEQ ID NO: 8;
a fifth primer set: SEQ ID NO: 9 and SEQ ID NO: 10;
a sixth primer set: SEQ ID NO: 11 and SEQ ID NO: 12;
a seventh primer set: SEQ ID NO: 12 and SEQ ID NO: 13;
an eighth primer set: SEQ ID NO: 14 and SEQ ID NO: 15; and
a ninth primer set: SEQ ID NO: 16, SEQ ID NO: 17, and SEQ ID NO: 18.

According to a second aspect, the present disclosure provides a kit for HLA gene sequencing, which includes said HLA gene amplifying primers.

According to a third aspect, the present disclosure provides a method of constructing an HLA gene sequencing library, including the following steps:
subjecting a test sample to amplification using the HLA gene amplifying primers to obtain an amplification product;
performing a ligation reaction between the amplification product and an adaptor to construct a library: and
purifying the library.

According to a fourth aspect, the present disclosure provides an HILA gene sequencing method, including constructing an HLA gene sequencing library using the method of constructing an HLA gene sequencing library, and then sequencing the library.

The present disclosure proposes nine primer sets, which respectively target different target sequences of the HLA gene. The nine primer sets may be used individually for PCR amplification to obtain amplified genes, or combinedly for single-tube multiplex PCR amplification. Using the nine primer sets, 11 HLA gene sequences (including DPB1, DQA1, DQB1, DPA1, DRB1/3/4/5, HLA-A, HLA-B, HLA-C) can be obtained by single-tube multiplex PCR amplification. Compared with current next-generation sequencing, which requires at least three tubes for separate amplification respectively to achieve the amplification of the above 11 gene sequences, the present disclosure has a more convenient experimental process and a more simplified process.

The library prepared with the amplification product produced using the primer set of the present disclosure can be used to detect new alleles based on the third-generation single-molecule real-time sequencing (SMRT sequencing) technology of Pacific Biosciences (PacBio), It has the advantages of long read length and high accuracy, solving the problem of inability of phasing, and can generally reach 3-field resolution without ambiguous results. The present disclosure, taking advantage of the characteristic of long read length of third-generation single-molecule sequencing technology, increases the amplification area with regard to the design of primers and improves the coverage of the amplification area. Traditional primers are designed for exon regions only, while the primer sets of the present disclosure can lead to amplification that yields not only information about exon regions but also more comprehensive information about introns. Combining the PacBio sequencing technology with the primer sets designed in the present disclosure. it can effectively provide complete HLA gene information, which provides a complete solution for further research on the function of HLA genes in the future, and improve the sensitivity and accuracy of typing.

The details of one or more embodiments of the present disclosure are set forth in the accompanying drawings and the description below. Other features, objects and advantages of the present disclosure will become apparent from the description, the accompanying drawings, and the claims.

### BRIEF DESCRIPTION OF THE DRAWINGS

In order to more clearly explain the implements of the present disclosure, the drawings in the embodiments will be briefly illustrated in the following. Obviously, the drawings as described in the following are only schematic diagrams of embodiments of the present disclosure. For those of ordinary skill in the art, other drawings can be obtained according to the provided drawings, without any creative efforts.

FIG. 1 is an electrophoretogram of some samples subjected to amplification using primers of an embodiment of the present disclosure.

FIG. 2 is an electrophoretogram of some samples subjected to amplification using primers of another embodiment of the present disclosure.

FIG. 3 is an electrophoretogram of some samples subjected to amplification using primers of another embodiment of the present disclosure.

FIG. 4 is an electrophoretogram of some samples subjected to amplification using primers of another embodiment of the present disclosure.

FIG. 5 is an electrophoretogram of some samples subjected to amplification using primers of another embodiment of the present disclosure,

FIG. 6 is an electrophoretogram of some samples subjected to amplification using primers of another embodiment of the present disclosure.

FIG. 7 is an electrophoretogram of some samples subjected to amplification using primers of another embodiment of the present disclosure.

FIG. 8 is an electrophoretogram of some samples subjected to amplification using primers of another embodiment of the present disclosure.

FIG. 9 is an electrophoretogram of some samples subjected to amplification using primers of another embodiment of the present disclosure.

FIG. 10 is a diagram of regions amplificated by primers of an embodiment of the present disclosure.

FIG. 11 is a figure showing the test result of the size of a library fragment according to an embodiment of the present disclosure.

### DETAILED DESCRIPTION OF THE EMBODIMENTS

In In order to facilitate the understanding of the present disclosure, the present disclosure will be described more fully hereinafter with reference to the related accompanying drawings. Preferable embodiments of the present disclosure are presented in the accompanying drawings. However, the present disclosure may be embodied in many different forms and is not limited to the embodiments described herein. Rather, these embodiments are provided so that the understanding of the content of the present disclosure will be more thorough.

All technical and scientific terms used herein have the same meaning as commonly understood by one of ordinary skill in the art to which the present disclosure applies, unless otherwise defined. The terms used in the specification of the present disclosure herein are for the purpose of describing specific embodiments only and are not intended to limit the present disclosure. The term “and/or” used herein includes any and all combinations of one or more of the associated listed items.

In the present disclosure, gDNA refers to genomic DNA, that is, the whole DNA of an organism in its haploid state.

In the present disclosure, the terms “target nucleic acid sequence”, “target nucleic acid” and “target sequence” refer to the objective nucleic acid sequence to be detected. In the present disclosure, the terms “target nucleic acid sequence”, “target nucleic acid” and “target sequence” have the same meaning and are used interchangeably. In the present disclosure, the terms “targeted sequence” and “target-specific sequence” refer to a sequence that is capable of selectively/specifically hybridizing to a target nucleic acid sequence under a condition that allows hybridization, annealing, or amplification of the nucleic acid, which includes a sequence complementary to the target nucleic acid sequence. In the present disclosure, the terms “targeted sequence” and “target-specific sequence” have the same meaning and are used interchangeably. It will be easily understood that a targeted sequence or target-specific sequence is specific for a target nucleic acid sequence.

In other words, under the condition that allows hybridization, annealing, or amplification of the nucleic acid, the targeted sequence or target-specific sequence hybridizes or anneals only to the specific target nucleic acid sequence, but not to other nucleic acid sequences,

In the present disclosure, “F” and “R”, the initial letters of “forward” and “reverse”, represent forward primer and reverse primer in the present disclosure, respectively. In the present disclosure, “forward primer” and “reverse primer” have the same meaning as “upstream primer” and “downstream primer”, respectively, and can be used interchangeably.

In the present disclosure, the term “upstream” is used to describe the relative positional relationship of two nucleic acid sequences (or two nucleic acid molecules), and bas the meaning as generally understood by those skilled in the art. For example, the expression “a nucleic acid sequence is located upstream of another nucleic acid sequence” means that, when aligned in the 5′ to 3′ direction, the former is located at a more forward position (i.e., closer to the 5′ end) than the latter. In the present disclosure, the term “downstream” has the opposite meaning to “upstream”.

In the present disclosure, the term “PCR” is the abbreviation of polymerase chain reaction and has the meaning generally understood by those skilled in the art, which refers to a reaction that amplifies a target nucleic acid using a nucleic acid polymerase and primers.

In the present disclosure, the term “multiplex PCR” refers to a PCR process that simultaneously amplifies multiple nucleic acid fragment products using two or more sets of primers in a single PCR system.

In the present disclosure, the terms “hybridization” and “annealing” mean a process by which complementary single-stranded nucleic acid molecules form a double-stranded nucleic acid. In the present disclosure, “hybridization” and “annealing” have the same meaning and are used interchangeably. Typically, two nucleic acid sequences that are completely complementary or substantially complementary may be subjected to hybridization or annealing. The complementarity required for hybridization or annealing of two nucleic acid sequences depends on the hybridization conditions used, particularly the temperature.

In the first aspect, the embodiments of the present disclosure provide HLA gene amplifying primers, including primers in any one or more of the following nine primer sets:
nucleotide sequences of the primers of the first primer set are: SEQ ID NO: 1 and SEQ ID NO: 2; nucleotide sequences of the primers of the second primer set are: SEQ ID NO: 3 and SEQ ID NO: 4; nucleotide sequences of the primers of the third primer set are: SEQ ID NO:5 and SEQ ID NO:6; nucleotide sequences of the primers of the fourth primer set are: SEQ ID NO:7 and SEQ ID NO:8; nucleotide sequences of the primers of the fifth primer set are: SEQ ID NO:9 and SEQ ID NO:10; nucleotide sequences of the primers of the sixth primer set are: SEQ ID NO:11 and SEQ ID NO:12; nucleotide sequences of the primers of the seventh primer set are: SEQ ID NO: 12 and SEQ ID NO: 13; nucleotide sequences of the primers of the eighth primer set are: SEQ ID NO: 14 and SEQ ID NO: 15; and nucleotide sequences of the primers of the ninth primer set are: SEQ ID NO:16. SEQ ID NO:17 and SEQ ID NO:18.

In some embodiments, the HLA gene amplifying primers include the primers in any two, three, four, five, six, seven, eight, or nine of the nine primer sets.

The present disclosure proposes nine primer sets, which respectively target different target sequences of the HLA gene. The nine primer sets may be used individually for PCR amplification to obtain amplification products of a certain target sequence, or combinedly for single-tube multiplex PCR amplification to obtain mixed amplification products of multiple target sequences. Using the nine primer sets. 11 HLA gene sequences (including DPB1, DQA1, DQB1, DPA1, DRB1/3/4/5, HLA-A, HLA-B. HLA-C) can be simultaneously amplified by single-tube multiplex PCR amplification. Compared with current next-generation sequencing, which requires at least three tubes for separate amplification respectively to achieve the simultaneous amplification of the above 11 gene sequences, the present disclosure has a more convenient experimental process and a more simplified process.

In multiplex PCR, multiple target sequences targeted by multiple primer sets are amplified simultaneously, and the amplified sequences are of different lengths, so there will inevitably be interference between the amplification of each gene fragment. Therefore. the adjustments of the ratio of each primer set and the ratio of primers in each primer set are of great significance to achieve the multiplex amplification with high quality.

In some embodiments, a molar ratio of the primers in each primer set is as follows:
the molar ratio of SEQ ID NO: 1 to SEQ ID NO: 2 is 1: (0.9˜1.1); the molar ratio of SEQ ID NO: 3 to SEQ ID NO: 4 is 1: (0.9˜1.1); the molar ratio of SEQ ID NO: 5 to SEQ ID NO: 6 is 1: (0.9˜1.1); the molar ratio of SEQ ID NO: 7 to SEQ ID NO: 8 is 1: (0.9˜1.1); the molar ratio of SEQ ID NO: 9 to SEQ ID NO: 10 is 1: (0.9˜1.1); the molar ratio of SEQ ID NO: 11 to SEQ ID NO: 12 is 1: (0.9˜1.1); the molar ratio of SEQ ID NO: 12 to SEQ ID NO: 13 is 1: (0.9˜1.1); the molar ratio of SEQ ID NO: 14 to SEQ ID NO: 15 is 1: (0.9˜1.1); and the molar ratio of SEQ ID NO:16, SEQ ID NO:17 and SEQ ID NO: 18 is (1.9˜2.1): (0.9˜1.1): (0.9˜1.1).

In some embodiments, the ratio of the sum of molars of the primers in each primer set is as follows: the first primer set:the second primer set:the third primer set:the fourth primer set:the fifth primer set:the sixth primer set:the seventh primer set:the eighth primer set:the ninth primer set=(8.9˜9.1):(5.4˜5.6):(21˜23):(1.4˜1.6):(1.8˜2):(0.9˜1.1):(2˜2.2):(0.9˜1.1):(2.4˜2.6).

In some embodiments, the sum of molars of the primers in each primer set is as follows:

| Primer | sum of mol |
|---|---|
| SEQ ID NO: 1 + SEQ ID NO: 2 | 8.9~9.1 |
| SEQ ID NO: 3 + SEQ ID NO: 4 | 5.4~5.6 |
| SEQ ID NO: 5 + SEQ ID NO: 6 | 21~23 |
| SEQ ID NO: 7 + SEQ ID NO: 8 | 1.4~1.6 |
| SEQ ID NO: 9 + SEQ ID NO: 10 | 1.8~2 |
| SEQ ID NO: 11 + SEQ ID NO: 12 | 0.9~1.1 |
| SEQ ID NO: 12 + SEQ ID NO: 13 | 2~2.2 |
| SEQ ID NO: 14 + SEQ ID NO: 15 | 0.9~1.1 |
| SEQ ID NO: 16 + SEQ ID NO: 17 + | 2.4~2.6 |
| SEQ ID NO: 18 | |

In some embodiments, while the primers in each primer set satisfy the above molar ratio, the sum of molars of the primers in each primer set can be the same multiple of the above sum of the molars.

For the PacBio third-generation sequencing platform, a simple and fast library construction and sequencing method of HLA gene has been studied. Taking the advantages of third-generation sequencing, combined with special primer amplification means, it can achieve the purpose of multiplex amplification at one time while overcoming the shortcomings of traditional sequencing methods.

In the second aspect, the embodiments of the present disclosure provide a kit for HLA gene sequencing, which includes the HLA gene amplifying primers described in any of the above embodiments. This kit is suitable for library construction and sequencing.

Optionally, the kit further includes any one or more of a PCR amplification reagent, a sequencing library construction reagent, and a gene purification reagent.

In some embodiments, the sequencing library construction reagent includes any one or more of an adaptor, an adaptor ligation reaction reagent, and an exonuclease.

The adaptor is a hairpin adaptor. By ligating the hairpin adaptors at both ends of a gene amplification fragment, the gene amplification fragment forms a circular closed structure.

In some embodiments, based on a 3.5 μL system, the adaptor ligation reaction reagent is a mixture of the following reaction reagents: 1 nmol of dNTP, 10 nmol of dATP, 0.75 U of T4 DNA polymerase, 2.5 U of T4 polynucleotide kinase, 120 U of T4 DNA ligase, T4 DNA polymerase buffer and water. 1 U of T4 DNA is defined as the amount of enzyme sufficient to incorporate 10 nmol of dNTP into an acid-insoluble precipitate within 30 minutes at 37° C.; IU of T4 polynucleotide kinase is defined as the amount of enzyme sufficient to incorporate 1 nmol of [γ-³²P] into an acid-insoluble precipitate within 30 minutes at 37° C.; and IU of T4 DNA ligase is defined as the amount of enzyme sufficient for more than 50% of the DNA fragments to be ligated a 6 μg of λDNA-HindIII digested materials is reacted for 30 minutes at 16° C. in a 20 μl ligation system. By rationally designing the formula and proportion of the adaptor ligation reaction reagents, this embodiment can realize one-step adaptor ligation, which improves the process efficiency compared with the traditional multi-step ligation.

In the third aspect, the embodiments of the present disclosure provide a method of constructing an HLA gene sequencing library, including the steps of:
subjecting a test sample to amplification using the HLA gene amplifying primers described in any of the above embodiments to obtain an amplification product;
performing a ligation reaction between the amplification product and an adaptor to construct a library: and
purifying the library.

In some embodiments, reaction conditions for the amplification are as follows: 2 min to 3 min holding at 93° C.˜95° C.; 30 cycles, each cycle including 10 seconds at 98° C.; 11 min to 13 min at 68° C. in each cycle from cycle 1 to cycle 10, with addition of 30 seconds per cycle starting from cycle 11; and 10 min holding at 68° C.

The primers are designed for the region to be amplified, because multiple pairs of primers may be needed to be used for amplification at the same time, and the lengths of the amplified sequences are different. By adjusting the ratio of primers and adjusting the appropriate reaction environment, multiplex amplification is achieved in a single tube.

In some embodiments, reaction conditions for the ligation reaction are as follows; 20 min to 30 min holding at 37° C.; 20 min to 30 min holding at 16° C. to 25° C.; and 10 min holding at 65° C.

The purification of the library is for the purpose of removing various reaction solvents and impurities introduced during the construction of the library.

In the fourth aspect, the embodiments of the present disclosure provide an HLA gene sequencing method, including constructing an HLA gene sequencing library using the method of constructing an HLA gene sequencing library described in any of the above embodiments and then sequencing the library.

In some embodiments, the sequencing is performed based on the PacBio sequencing platform.

Pacbio's third-generation sequencing is based on the principle of sequencing by synthesis, to perform sequencing reactions using the single-molecule real-time sequencing (SMRT sequencing) system chip as a carrier, The fundamentals are as follows: a polymerase captures a library DNA sequence and is anchored at the bottom of a zero mode waveguide; four different fluorescently-labeled dNTPs randomly enter the bottom of the zero mode waveguide; the fluorescent dNTPs are irradiated by laser, allowing the fluorescence to be emitted and detected; a fluorescent dNTP, which matches a base of the DNA template, is incorporated for extension under the action of the enzyme; during the reaction of the enzyme, it, on the one hand, extends the chain and on the other hand causes the fluorescent group on the dNTP to fall off: as the polymerization reaction occurs, the sequencing is performed simultaneously,

In Pacbio's third-generation sequencing, DNA molecules are ligated with a hairpin adapter, and thus the whole constructed library is a circular molecule conducive to Its repeated replication. Moreover, repeated sequencing of one fragment can improve the accuracy, unlike the Illumina sequencing, in which noise may be caused and limit the read length due to phasing and prephasing caused by measuring multiple bases at the same time.

With the Pacific Biosciences sequencing platform, complete information can be obtained without the need to assemble the raw data obtained. This avoids problems such as the inability to distinguish sister chromosome information through first and second-generation sequencing technologies and possible error information caused by assembly. taking the entire HLA genotyping technique to a new level.

In some embodiments, the test samples suitable for the sequencing method are from pure DNA samples or other sample forms, such as whole blood, plasma, and the like.

The following are specific examples.

First, the primers are designed for the region to be amplified, because multiple pairs of primers may be needed to be used for amplification at the same time, and the lengths of the amplified sequences are different. By adjusting the ratio of primers. finding suitable reagents, and adjusting the appropriate reaction environment, multiplex amplification is achieved in a single tube. Moreover, based on the characteristic of long read length in the third-generation single-molecule real-time sequencing technology, a library is constructed directly by the Pacific Biosciences third-generation sequencing using the amplified products, and then the amplified HLA genotyping information is obtained with the Pacific Biosciences sequencing platform. The entire process is divided into five parts, in order of amplification, library construction, purification, library mixing, and computer-sequencing.

### Example 1

### I. Amplification

Amplification was performed using 88 human gDNA standard samples purchased from Coriell. It was proved by the results of a large number of samples that the methods of the present disclosure have universality and sensitivity.

Specific amplifying primers were designed for different HLA loci and 5′-phosphorylated primers were synthesized. Sequences of the primers are shown in the following Table 1:

TABLE 1

| No. | Primer (barcode-H) | Sequence |
|---|---|---|
| SEQ ID NO: 1 | HLA-DQA1-F-H | gggttcagacatgatgactgagc |
| SEQ ID NO: 2 | HLA-DQA1-R-H | ggatttagggttctttgcaccaaac |
| SEQ ID NO: 3 | HLA-DQB1-F-H | tcatgtgcttctcttgagcagtctga |
| SEQ ID NO: 4 | HLA-DQB1-R-H | trtgacagcaattttctctcccct |
| SEQ ID NO: 5 | HLA-DPB1-F-H | cagactctgtccaatcccagggtcaca |
| SEQ ID NO: 6 | HLA-DPB1-R-H | gagcacagtagctttcgggaattgacca |
| SEQ ID NO: 7 | HLA-DPA1-F-H | gaaaacatttgccgtcagaagaaggtg |
| SEQ ID NO: 8 | HLA-DPA1-R-H | ccttttgtacctgaggtccagcatgaa |
| SEQ ID NO: 9 | HLA-A-F-H | atcctggatactcacgacgcggac |
| SEQ ID NO: 10 | HLA-A-R-H | catcaacctctcatggcaagaattt |
| SEQ ID NO: 11 | HLA-B-F-H | tgaatggctctgaaaatttgtctcagaa |
| SEQ ID NO: 12 | HLA-C-R-H | ggggtctctccctggtttccacaga |
| SEQ ID NO: 13 | HLA-C-F-H | tgamtgggtctgaaaatttgtctcagaa |
| SEQ ID NO: 14 | HLA-DRB-E1-F-H | ctctccattgcayataggtggcccc |
| SEQ ID NO: 15 | HLA-DRB-E1-R-H | atgcttwttgggttagtgtctgtctcctc |
| SEQ ID NO: 16 | HLA-DRB-E2E6-F-H | caghrgastggagaggtctgttttcc |
| SEQ ID NO: 17 | HLA-DRB-E2E6-R1-H | tctkcayttcagctcaggaatcctgca |
| SEQ ID NO: 18 | HLA-DRB-E2E6-R2-H | tctgcacttcagctcamgagtcctgca |

SEQ ID NOS: 1, 3, 5, 7, 9, 11, 13, 14, and 16 were forward primers, and SEQ ID NOS: 2, 4, 6, 8, 10, 12, 15, 17, and 18 were reverse primers. The primers were prepared and diluted using an Elution Buffer. First, the forward and reverse primers were diluted separately, and then mixed. Next, a primer MIX containing all 18 primers was formulated according to the primer volume in Table 2 below.

TABLE 2

| | | Amplified | Primer |
|---|---|---|---|
| | | fragment size | volume |
| | Gene primer | (bp) | (ul) |
| 1 | HLA-DPB1-F/R(20 uM) | 11774 | 1.1 |
| 2 | HLA-DQA1-F/R(5 uM) | 8657 | 1.8 |
| 3 | HLA-DQB1F/R(5 uM) | 7118 | 1.1 |
| 4 | HLA-DPA1-F/R(5 uM) | 5985 | 0.3 |
| 5 | DRB-E2E6-F1/R1/R2 | 4.9-6.4k | 0.5 |
| | (5 uM) = 2:1:1 | | |
| 6 | HLA-B-F/-C-R(5 uM) | 3938 | 0.2 |
| 7 | HLA-C-F/R(5 uM) | 3969 | 0.42 |
| 8 | HLA-A-F/R(5 uM) | 3398 | 0.38 |
| 9 | DRB-E1-F/R(5 uM) | 2800 | 0.2 |

After the primer MIX was formulated, the reagents were added to a new PCR tube In sequence according to the PCR amplification system in Table 3 below, with the tube lid closed finally. The amplification system was mixed well and centrifuged.

TABLE 3

| | Single sample |
|---|---|
| KOD Neo FX Buffer | 12.5 μL |
| dNTPs | 5 μL |
| Total Primer MIX | 6 μL |
| gDNA | 1 μL |
| KOD Neo FX | 0.5 μL |

The PCR program was set according to the parameters in Table 4 below for amplification, Hot Lid: 105° C., rising and cooling rate: 2.0° C./s.

TABLE 4

| Temperature | Time | Cycles |
|---|---|---|
| 94° C. | 2 min | 1× |
| 98° C. | 10 s | 30× |
| 68° C. | 12 min in each cycle from cycle 1 to 10; | |
| | with addition of 30 seconds per cycle | |
| | starting from cycle 11 | |
| 68° C. | 10 min | 1× |
| 8° C. | ∞ | |

The amplified products were electrophoresed using 1% agarose gel to determine whether the target gene was amplified. The conditions of agarose gel electrophoresis were as follows: voltage: 150 V, time: 50 min, sample volume: 5 ul of amplification product+5 ul of 6×loading buffer. The amplification results of gDNA purchased from Coriell are shown in FIGS. 1 to 9.

FIGS. 1-9 shows the results of agarose gel electrophoresis with Vazyme DL5000 as the Marker. The poorly amplified samples were amplified again. The bands in each lane in the figure were products respectively obtained by amplification with the above primer Mix using gDNA purchased from Coriell as the template. The following numbers represent the accession numbers of each gDNA sample on Coriell:

FIG. 1. (D1NA16689 (2) NA17212 (3) NA17272 (4) NA17222 (S) NA17283 (6) NA17218 (7) NA1721S (8) NA17119 (9) NA17084 (10) NA17466 (11) NA17240 (12) NA17292 (13) NA17231 (14) NA17263 (15) NA17201.

FIG. 2. (1) NA17249 (2) NA17618 (3) NA17230 (4) NA17269 (5) NA17129 (6) NA17130 (7) NA17264 (8) NA17203 (9) NA17247 (10) NA17242 (ID) NA17438 (12) NA17262 (13) NA02016 (14) NA 17236 (15) NA17217.

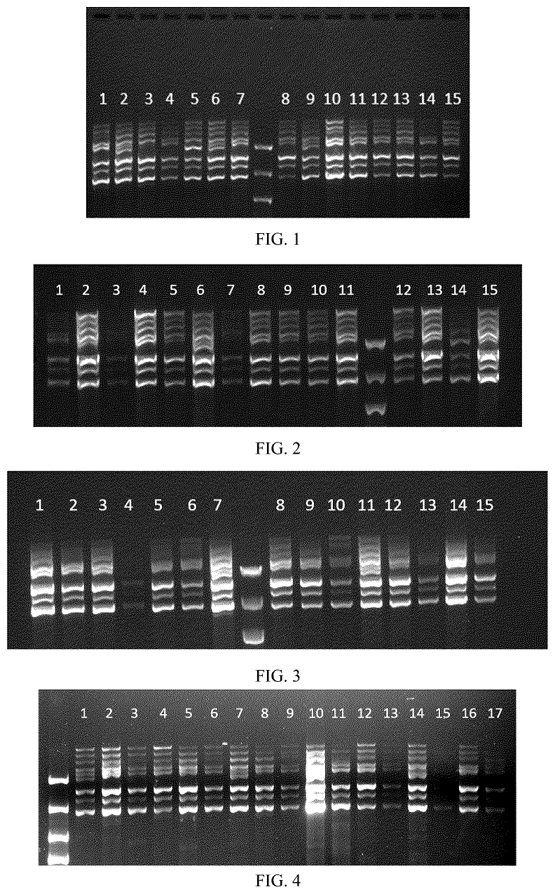

FIG. 3. (1) NA17286 (2) NA17248 (3) NA17282 (4) NA17052 (5) NA17019 (6) NA17291 (7) NA17235 (8) NA16654 (9) NA17287 (10) NA17275 (11) NA17261 (12) NA17216 (13) NA17256 (14) NA 17286 (IS) NA17221.

FIG. 4. (1) NA17075 (2) NA17224 (3) NA13000 (4) NA17039 (5) NA17281 (6) NA12244 (7) NA17206 (8) NA17245 (9) NA17208 (10) NA17293 (11) NA17274 (12) NA17234 (13) NA 17265 (14) NA17276 (15) NA17289 (16) NA17114 (17) NA17237.

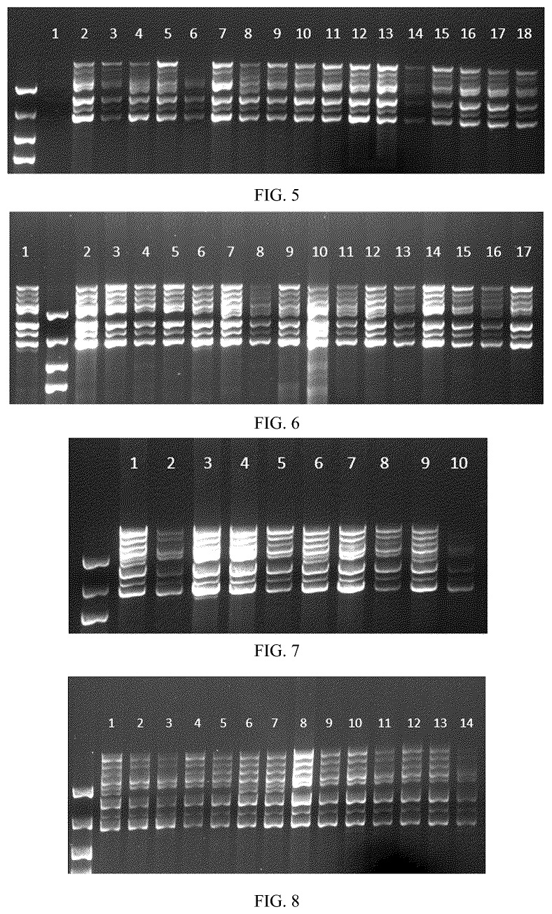

FIG. 5. (1) NA17279 (2) NA23090 (3) NA17228 (4) NA07439 (5) NA17298 (6) NA17229 (7) NA17248 (8) NA17214 (9) NA16688 (10) NA17254 (11) NA23093 (12) NA17207 (13) NA17246 (14) NA17209 (15) NA17073 (16) NA17285 (17) NA12273 (18) NA17260.

FIG. 6. (1) NA17115 (2) NA17204 (3) NA17243 (4) NA17267 (5) NA17058 (6) NA17232 (7) NA17075 (8) NA17295 (9) NA17268 (10) NA17440 (11) NA17290 (12) NA 17296 (13) NA 17226 (14) NA 17257 (15) NA17280 (16) NA10005 (17) NA17213.

FIG. 7. (1) NA17261 (2) NA09301 (3) NA17219 (4) NA17227 (5) NA17287 (6) NA17244 (7) NA17288 (8) NA17234 (9) NA17211 (10) NA17256.

FIG. 8. (1) NA17298 (2) NA17291 (3) NA09301 (4) NA17262 (5) NA17242 (6) NA17214 (7) NA17206 (8) NA17269 (9) NA17230 (10) NA17438 (11) NA17114 (12) NA17073 (13) NA17247 (14) NA17229.

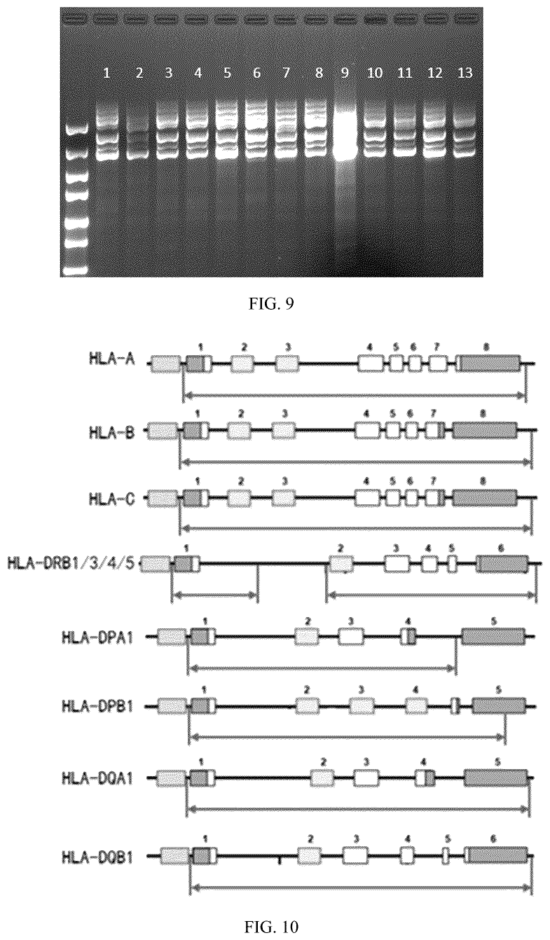

FIG. 9. (1) NA17264 (2) NA17236 (3) NA17265 (4) NA17237 (5) NA17233 (6) NA17277 (7) NA17057 (8) NA17020 (9) NA17205 (10) NA17220 (11) NA17209 (12) NA17087 (13) NA17212.

FIG. 10 shows the regions amplified using nine primer sets.

### II. Library Construction

After the amplification, the amplified product was vibrated and shortly spun, and then stood horizontally after the spin. Then an adaptor ligation was performed as follows.

According to the traditional PacBio adaptor annealing program, the Barcode adaptor was subjected to annealing for future use.

The Ligase Mix was formulated according to Table 5 below.

TABLE 5

| Reaction reagent | Single sample |
|---|---|
| 10 × T4 DNA ligase buffer | 1.0 μL |
| 10 mM dNTP | 0.1 μL |
| ATP Solution (100 mM) | 0.1 μL |
| T4 DNA polymerase | 0.25 μL |
| T4 Polynucleotide Kinase | 0.25 μL |
| T4 DNA ligase | 0.3 μL |
| DNasc/RNase-Free H₂O | 1.5 μL |
| Total | 3.5 μL |

A new PCR tube was used to formulate a ligation system according to Table 6 below.

TABLE 6

| | |
|---|---|
| Amplification product | 4 μL |
| Barcode adaptor | 2.5 μL |
| Ligase Mix | 3.5 μL |
| Total | 10 μL |

Once formulation was completed, the tube lid was closed. The tube was shortly spun, vibrated for well mix, and then shortly spun again, and then put into a PCR instrument pre-set according to Table 7 for the ligation reaction, and finally, the ligation product was obtained;

Reaction procedure: hot lid: 75° C., rising and cooling rate: 2.5° C./s.

TABLE 7

| Temperature | Time | Cycles |
|---|---|---|
| 37° C. | 25 min | 1× |
| 25° C. | 20 min | 1× |
| 65° C. | 10 min | 1× |
| 4° C. | ∞ | |

After the ligation reaction, the exonuclease Mix was prepared according to Table 8 below.

TABLE 8

| | Single sample |
|---|---|
| Exonuclease 1 | 0.5 μL |
| Exonuclease 3 | 0.5 μL |
| Total | 1 μL |

Tubes containing the ligation products were placed on ice and 1 μl of exonuclease Mix was added to each tube.

The tube lid was closed. The tube was shortly spun, vibrated for well mix, and then shortly spun again, and then put into a PCR instrument pre-set according to Table 7 for a digestion reaction.

Reaction procedure: hot lid: 45° C., rising and cooling rate: 2.5° C./s.

TABLE 9

| Temperature | Time | Cycle |
|---|---|---|
| 37° C. | 60 min | 1× |
| 4° C. | ∞ | |

### III. Purification

AMPure® magnetic beads (AMPure® PB beads) were taken out of the refrigerator half an hour in advance, vibrated for resuspension, and then placed on a vertical mixer for equilibration at room temperature for 30 min.
1. 6.6 μL. of magnetic beads (0.6×) were added to the products digested by the exonuclease, and mixed well by gently flicking the tube wall or by vibrating at low speed. The tube was shortly spun and left at room temperature for 10 min, during which the tube wall was gently flicked for 2-3 times:
2. The PCR tube was short spun, then placed on a magnetic grate to adsorb the magnetic beads for 10 min, during which 70% alcohol (which needed to be prepared freshly) was prepared:
3. The supernatant was discarded, and then 200 μL of 70% alcohol was added along the side of the tube wall opposite to the side where the magnetic beads are adsorbed, so as not to flush down the magnetic beads;
4. The previous step was repeated for a second alcohol cleaning;
5. The alcohol was discarded, the PCR tube was centrifuged and put back on the magnetic grate, and then the residual liquid was discarded;
6. The PCR tube was opened and dried for no more than 30 s, and then 20 μL of Elution Buffer (EB) was added:
7. The magnetic beads were vibrated for suspension and kept mixed in the system. the tube was placed at room temperature for 10 min for DNA elution, during which the tube wall was gently flicked 2-3 times;
8. The PCR tube was shortly spun, then placed on a magnetic grate to adsorb the magnetic beads for 10 min;
9. 20 μL of the supernatant was pipetted to a new PCR tube;
10. A single sample library was quantified using a Qubit Flex Quantitation Kit (Thermo Fisher), For 88 samples, the same mass of 3 ng library DNA was taken from each single sample library to form a mixed library.

### IV. Library Mixing

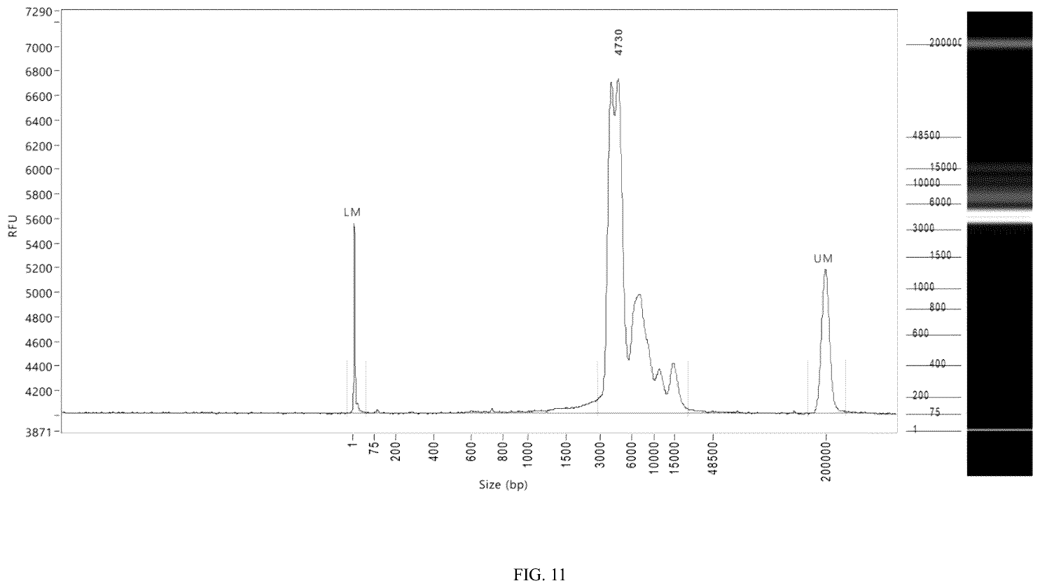

1. 204 μL (3 ng) of the mixed library was accurately measured with a pipette and added to a new EP tube, then 122.4 μL of PB beads (0.6×) was added, mixed well by gently flicking the tube wall or by vibrating at low speed. The tube was shortly spun and left at room temperature for 10 min, during which the tube wall was gently flicked for 2-3 times;
2. The EP tube was shortly spun, then placed on a magnetic grate to adsorb the magnetic beads for 10 min;
3. The supernatant was discarded, and then 200 μl of 70% alcohol was added along the side of the tube wall opposite to the side where the magnetic beads are adsorbed, so as not to flush down the magnetic beads;
4. The previous step was repeated for a second alcohol cleaning;
5. The alcohol was discarded, the EP tube was centrifuged and put back on the magnetic grate, and then the residual liquid was discarded;
6. The EP tube was opened and dried for no more than 30 s, and then 15 μL of EB was added—the volume of EB can be adjusted properly according to the number of samples and the volume of beads;
7. The magnetic beads were vibrated for suspension, and the tube was placed at room temperature for 10 min for DNA elution, during which the tube wall was gently flicked 2-3 times;
8. The EP tube was shortly spun, then placed on a magnetic grate to adsorb the magnetic beads for 5 min;
9. All supernatant was pipetted to a new PCR tube;
10. The final library was quantified using the Qubit Flex Quantitation Kit (Thermo Fisher), which was 14 ng/ul in concentration.
11. The library was tested using the 5200 Fragment Analyzer System, and calculated for the average fragment size, and the analysis results are shown in FIG. 11. The relationship between fragment size and number as illustrated by the results was consistent with that of the actual samples.

### V. Computer-Sequencing

Computer-sequencing was performed according to the process of computer-sequencing of amplicons recommended by PacBio Sequel 11. The results are shown in Tables 10-12 below.

TABLE 10

| | | | | |
|---|---|---|---|---|
| Sample | Typing | HLA-A | HLA-B | HLA-C |

| | | | | | | | |
|---|---|---|---|---|---|---|---|
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |

[symbol]

indicates data missing or illegible when filed

TABLE 11

| Sample | Typing | HLA-DPA1 | | HLA-DPB1 | | HLA-DQA1 | |
|---|---|---|---|---|---|---|---|
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |

[symbol]

indicates data missing or illegible when filed

TABLE 12

| | | | | |
|---|---|---|---|---|
| Sample | Typing | HLA-DQB1 | [symbol] | [symbol] |

| | | | | | | | |
|---|---|---|---|---|---|---|---|
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | | |
| | [symbol] | [symbol] | [symbol] | [symbol] | [symbol] | | |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | | |
| | [symbol] | [symbol] | [symbol] | [symbol] | [symbol] | | |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | | |
| | [symbol] | [symbol] | [symbol] | [symbol] | [symbol] | | |
| [symbol] | [symbol] | [symbol] | [symbol] | [symbol] | [symbol] | [symbol] | |
| | [symbol] | [symbol] | [symbol] | [symbol] | [symbol] | [symbol] | [symbol] |
| [symbol] | [symbol] | [symbol] | [symbol] | [symbol] | [symbol] | | |
| | [symbol] | [symbol] | [symbol] | [symbol] | [symbol] | | |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] |

[symbol]

indicates data missing or illegible when filed

Note: the data in hold were HLA genes with improved typing accuracy by the present product compared to the reference typing accuracy.

The results of HLA genotyping by third-generation sequencing are summarized in Table 13.

TABLE 13
HLA Typing Results

| | | Data | | Correct | |
|---|---|---|---|---|---|
| | Reference | Correctly | Capture | Number of | Capture |
| | Data | Captured | Sensitivity | Typing | Accuracy |
| HLA-A | 176 | 176 | 100% | 176 | 100% |
| HLA-B | 176 | 176 | 100% | 176 | 100% |
| HLA-C | 176 | 176 | 100% | 176 | 100% |
| HLA-DPA1 | 176 | 176 | 100% | 176 | 100% |
| HLA-DPB1 | 176 | 176 | 100% | 176 | 100% |
| HLA-DQA1 | 176 | 176 | 100% | 176 | 100% |
| HLA-DQB1 | 176 | 176 | 100% | 176 | 100% |
| HLA-DRB1 | 176 | 176 | 100% | 176 | 100% |
| HLA-DRB3/4/5 | 168 | 168 | 100% | 168 | 100% |

The technical features of the above embodiments can be combined arbitrarily. In order to make the description concise, not all possible combinations of the technical features in the above embodiments are described. However, as long as there is no contradiction in the combination of these technical features, all should be considered as the scope described in this specification.

The aforementioned embodiments only illustrate several embodiments of the present disclosure, which facilitate a specific and detailed understanding of the technical solutions of the present disclosure, but they cannot be understood to limit the protection scope of the present disclosure. It should be noted that a plurality of variations and modifications may be made by those skilled in the art without departing from the conception of the present disclosure, which are all within the scope of protection of the present disclosure. Accordingly, the scope of protection of the present disclosure shall be based on the appended claims, and the description may be used to interpret the content of the claims.

1. HLA gene amplifying primers, comprising primers in any one or more of the following nine primer sets:
a first primer set: SEQ ID NO: 1 and SEQ ID NO: 2;
a second primer set: SEQ ID NO: 3 and SEQ ID NO: 4;
a third primer set: SEQ ID NO: 5 and SEQ ID NO: 6;
a fourth primer set: SEQ ID NO: 7 and SEQ ID NO: 8;
a fifth primer set: SEQ ID NO: 9 and SEQ ID NO: 10;
a sixth primer set: SEQ ID NO: 11 and SEQ ID NO: 12;
a seventh primer set: SEQ ID NO: 12 and SEQ ID NO: 13;
an eighth primer set: SEQ ID NO: 14 and SEQ ID NO: 15; and
a ninth primer set: SEQ ID NO: 16, SEQ ID NO: 17, and SEQ ID NO: 18.

2. The HLA gene amplifying primers according to claim 1, comprising the primers in any two, three, four, five, six, seven, eight, or nine of the nine primer sets.

3. The HLA gene amplifying primers according to claim 1, wherein a molar ratio of the primers in each primer set is as follows:
the molar ratio of SEQ ID NO: 1 to SEQ ID NO: 2 is 1:(0.9˜1.1);
the molar ratio of SEQ ID NO: 3 to SEQ ID NO: 4 is 1:(0.9˜1.1);
the molar ratio of SEQ ID NO: 5 to SEQ ID NO: 6 is 1:(0.9˜1.1);
the molar ratio of SEQ ID NO: 7 to SEQ ID NO: 8 is 1:(0.9˜1.1);
the molar ratio of SEQ ID NO: 9 to SEQ ID NO: 10 is 1:(0.9˜1.1);
the molar ratio of SEQ ID NO: 11 to SEQ ID NO: 12 is 1:(0.9˜1.1);
the molar ratio of SEQ ID NO: 12 to SEQ ID NO: 13 is 1:(0.9˜1.1);
the molar ratio of SEQ ID NO: 14 to SEQ ID NO: 15 is 1:(0.9˜1.1);
the molar ratio of SEQ ID NO: 16 to SEQ ID NO: 17 is 18: (1.9˜2.1):(0.9˜1.1): (0.9˜1.1).

4. The HLA gene amplifying primers according to claim 3, wherein the ratio of the sum of molars of the primers in each primer set is as follows: the first primer set:the second primer set:the third primer set:the fourth primer set:the fifth primer set:the sixth primer set:the seventh primer set:the eighth primer set:the ninth primer set=(8.9˜9.1):(5.4˜5.6):(21˜23):(1.4˜1.6):(1.8˜2):(0.9˜1.1):(2˜2.2):(0.9˜1.1):(2.4˜2.6).

5. A kit for HLA gene sequencing, comprising HLA gene amplifying primers of claim 1.

6. The kit according to claim 5, further comprising any one or more of a PCR amplification reagent, a sequencing library construction reagent, and a gene purification reagent.

7. The kit according to claim 6, wherein the sequencing library construction reagent comprises any one or more of an adaptor, an adaptor ligation reaction reagent, and an exonuclease.

8. The kit according to claim 7, wherein based on a 3.5 μL system, the adaptor ligation reaction reagent is a mixture of the following reaction reagents: 1 nmol of dNTP, 10 nmol of dATP, 0.75 U of T4 DNA polymerase, 2.5 U of T4 polynucleotide kinase, 120 U of T4 DNA ligase, T4 DNA polymerase buffer and water.

9. A method of constructing an HLA gene sequencing library, comprising the steps of:
subjecting a test sample to amplification using HLA gene amplifying primers of claim 1 to obtain an amplification product;
performing a ligation reaction between the amplification product and an adaptor to construct a library; and
purifying the library.

10. The method according to claim 9, wherein reaction conditions for the amplification are as follows:
2 min to 3 min holding at 93° C.˜95° C.;
30 cycles, each cycle including 10 seconds at 98° C.; 11 min to 13 min at 68° C. in each cycle from cycle 1 to cycle 10, with addition of 30 seconds per cycle starting from cycle 11; and
10 min holding at 68° C.

11. The method according to claim 9, wherein reaction conditions for the ligation reaction are as follows: 20 min to 30 min holding at 37° C.; 20 min to 30 min holding at 16° C. to 25° C.; and 10 min holding at 65° C.

12. An HLA gene sequencing method, comprising constructing an HLA gene sequencing library using a method of claim 9, and then sequencing the library.

13. The HLA gene sequencing method according to claim 12, wherein the sequencing is performed based on a PacBio sequencing platform.


SEQUENCE LISTING (18 sequences)

SEQ ID NO: 1 - DNA, 23 bp, Artificial Sequence
  HLA-DQA1-F-H
gggttcagacatgatgactgagc

SEQ ID NO: 2 - DNA, 25 bp, Artificial Sequence
  HLA-DQA1-R-H
ggatttagggttctttgcaccaaac

SEQ ID NO: 3 - DNA, 26 bp, Artificial Sequence
  HLA-DQB1-F-H
tcatgtgcttctcttgagcagtctga

SEQ ID NO: 4 - DNA, 24 bp, Artificial Sequence
  HLA-DQB1-R-H
trtgacagcaattttctctcccct

SEQ ID NO: 5 - DNA, 27 bp, Artificial Sequence
  HLA-DPB1-F-H
cagactctgtccaatcccagggtcaca

SEQ ID NO: 6 - DNA, 28 bp, Artificial Sequence
  HLA-DPB1-R-H
gagcacagtagctttcgggaattgacca

SEQ ID NO: 7 - DNA, 27 bp, Artificial Sequence
  HLA-DPA1-F-H
gaaaacatttgccgtcagaagaaggtg

SEQ ID NO: 8 - DNA, 27 bp, Artificial Sequence
  HLA-DPA1-R-H
ccttttgtacctgaggtccagcatgaa

SEQ ID NO: 9 - DNA, 24 bp, Artificial Sequence
  HLA-A-F-H
atcctggatactcacgacgcggac

SEQ ID NO: 10 - DNA, 25 bp, Artificial Sequence
  HLA-A-R-H
catcaacctctcatggcaagaattt

SEQ ID NO: 11 - DNA, 28 bp, Artificial Sequence
  HLA-B-F-H
tgaatggctctgaaaatttgtctcagaa

SEQ ID NO: 12 - DNA, 25 bp, Artificial Sequence
  HLA-C-R-H
ggggtctctccctggtttccacaga

SEQ ID NO: 13 - DNA, 28 bp, Artificial Sequence
  HLA-C-F-H
tgamtgggtctgaaaatttgtctcagaa

SEQ ID NO: 14 - DNA, 25 bp, Artificial Sequence
  HLA-DRB-E1-F-H
ctctccattgcayataggtggcccc

SEQ ID NO: 15 - DNA, 29 bp, Artificial Sequence
  HLA-DRB-E1-R-H
atgcttwttgggttagtgtctgtctcctc

SEQ ID NO: 16 - DNA, 26 bp, Artificial Sequence
  HLA-DRB-E2E6-F-H
caghrgastggagaggtctgttttcc

SEQ ID NO: 17 - DNA, 27 bp, Artificial Sequence
  HLA-DRB-E2E6-R1-H
tctkcayttcagctcaggaatcctgca

SEQ ID NO: 18 - DNA, 27 bp, Artificial Sequence
  HLA-DRB-E2E6-R2-H
tctgcacttcagctcamgagtcctgca